(19) United States
(12) Patent Application Publication
(10) Pub. No.: US 2017/0035268 A1
(43) Pub. Date: Feb. 9, 2017

(54) STEREO DISPLAY SYSTEM AND METHOD FOR ENDOSCOPE USING SHAPE-FROM-SHADING ALGORITHM

(71) Applicant: MING SHI CO., LTD., Changhua City (TW)

(72) Inventors: Atul Kumar, Lukang Township (TW); Yen-Yu WANG, Lukang Township (TW); Kai-Che LIU, Lukang Township (TW); Min-Liang WANG, Lukang Township (TW); Ching-Jen WU, New Taipei City (TW)

(73) Assignee: MING SHI CO., LTD., Changhua City (TW)

(21) Appl. No.: 14/821,042

(22) Filed: Aug. 7, 2015

(51) Int. Cl. A61B 1/00; A61B 1/06; A61B 1/04

(52) U.S. Cl. CPC A61B 1/00009; A61B 1/04; A61B 1/00045; A61B 1/0661

(57) ABSTRACT

A stereo display system using shape from shading algorithm is an image conversion device connected between a monoscopic endoscope and a 3D monitor. The system applies the algorithm which generates a depth map for a 2D image of video frames. The algorithm first calculates a direction of a light source for the 2D image and based upon the information of light distribution and shading for the 2D image to generate the depth map. The depth map is used to calculate another view of the original 2D image by depth image based rendering algorithm in generation of stereoscopic images. After the new view is rendered, the stereo display system also needs to convert the display format of the stereoscopic images for different kinds of 3D displays. Base on this method, it is necessary to replace the whole monoscopic endoscope with a stereo-endoscope system and no modification is required for the monoscopic endoscope.

### BACKGROUND OF THE INVENTION

1. Field of the Invention

The present invention relates to a stereo display system for endoscope and, more particularly, to a stereo display system for endoscope using shape-from-shading algorithm to generate stereo images.

2. Description of the Related Art

Minimally invasive surgery has become an indispensable part in surgical treatment of current medical behavior and can be performed by endoscope-assisted surgical instruments to allow smaller incision and less tissue trauma, thereby shortening patient's recovery cycle and reducing overall medical expense. However, conventional minimally invasive surgery all employs monoscopic endoscope, which only displays two-dimensional (2D) images lacking depth information. Therefore, it is challenging for a surgeon to accurately move surgical instruments to a correct location inside a patient's body. Surgeons usually perceive depth in 2D images according to motion parallax, monocular cues and other indirect evidences for positioning accuracy. Providing stereo images capable of directly providing depth perception without going through additional means, such as motion parallax, monocular cues and other indirect evidences, is still the best approach in resolving the conventional inaccurate positioning issue at the cost of a dual-camera endoscope. Despite the advantages of depth information or stereo images required by surgeons, the dual-camera endoscope has the drawback of being much more expensive than the monoscopic endoscope and is less accepted accordingly.

### SUMMARY OF THE INVENTION

An objective of the present invention is to provide a stereo display system and a stereo display method using shape-from-shading algorithm capable of providing stereoscopic images with a monoscopic endoscope through the shape-from-shading algorithm.

To achieve the foregoing objective, the stereo display system for endoscope using shape-from-shading algorithm includes a monoscopic endoscope, a three-dimensional (3D) display, and an image conversion device.

The monoscopic endoscope captures the two-dimensional (2D) images.

The image conversion device is connected between the monoscopic endoscope and the 3D display and has an input port for endoscope and a 2D-to-3D conversion unit.

The input port for endoscope is connected to the monoscopic endoscope to receive the 2D image from the monoscopic endoscope.

The 2D-to-3D conversion unit applies shape from shading algorithm adapted to calculate a direction of a light source for the 2D image, and calculates a depth map based upon information of light distribution and shading of the 2D image, and applies depth image based rendering algorithm to convert the 2D image to a stereoscopic image with the information of light distribution and shading of the 2D image.

The image output port is connected with the 2D-to-3D image conversion unit and the 3D display to receive the stereo images and display the stereo image on the 3D display.

To achieve the foregoing objective, the stereo display method for endoscope using shape-from-shading algorithm includes steps of:

capturing a two-dimensional (2D) image, wherein an image-capturing unit is used to acquire a 2D image from a monoscopic endoscope with illumination from a light source;

calculating a light direction and a camera position for the 2D image;

generating a depth map of the 2D image using shape-from-shading method, wherein the shape-from-shading method combines the light direction and an iterative approach to solve equations involving a gradient variation of pixel intensity values in the 2D image; and

generating a stereoscopic image by combining the depth map and the 2D image.

Given the foregoing stereo display system and method using shape-from-shading algorithm, the 2D image taken by the monoscopic endoscope is processed by the shape-from-shading algorithm to calculate depth information in generation of a depth map, and the 2D image along with the depth map form the stereoscopic image that is outputted to the 3D display for users to view the converted stereoscopic image. As there is no need to replace a monoscopic endoscope with a dual-lens endoscope and modify the hardware structure of the existing monoscopic endoscope, the issues of no stereoscopic image available to monoscopic endoscope and costly dual-lens endoscope encountered upon the demand of stereoscopic images can be resolved.

Other objectives, advantages and novel features of the invention will become more apparent from the following detailed description when taken in conjunction with the accompanying drawings.

### BRIEF DESCRIPTION OF THE DRAWINGS

FIG. 1 is a functional block diagram of a stereo display system for endoscope using shape-from-shading algorithm in accordance with the present invention; and

FIG. 2 is a flow diagram of a stereo display method for endoscope using shape-from-shading algorithm in accordance with the present invention.

### DETAILED DESCRIPTION OF THE INVENTION

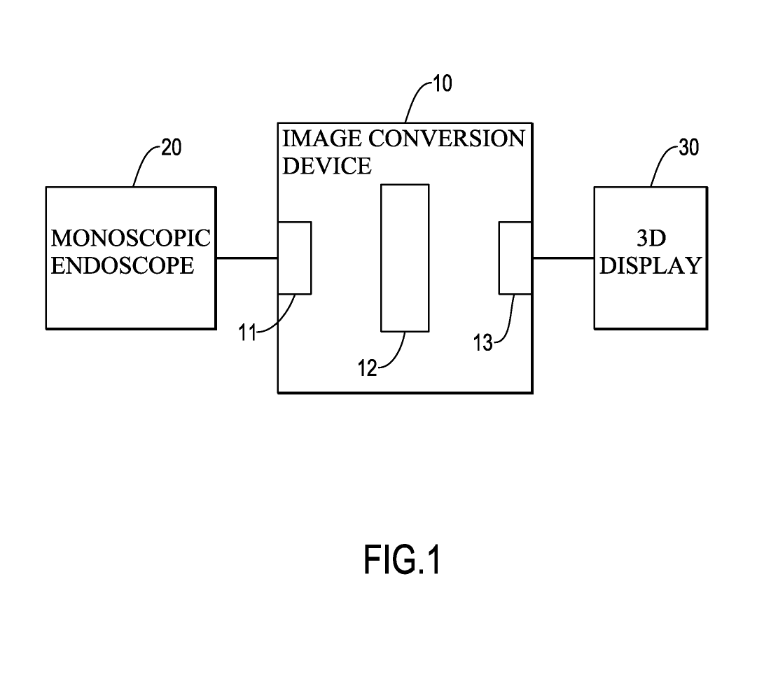

With reference to FIG. 1, a stereo display system for endoscope using shape-from-shading algorithm in accordance with the present invention includes a monoscopic endoscope 20, a three-dimensional (3D) display 30, and an image conversion device 10.

The image conversion device 10 is connected between the monoscopic endoscope 20 and the 3D display 30, and has an input port for endoscope 11, a 2D-to-3D image conversion unit 12, and an image output port 13. The input port for endoscope 11 is connected to the monoscopic endoscope 20. The 2D-to-3D image conversion unit 12 is electrically connected to the input port for endoscope 11, acquires a 2D image from the monoscopic endoscope 20, generates a depth map of the 2D image, and converts the 2D images and the depth map into a stereoscopic image using shape-from-shading algorithm built in the 2D-to-3D image conversion unit 12. The image output port 13 is electrically connected to the 2D-to-3D image conversion unit 12, is connected to the 3D display 30, and outputs the stereoscopic image to the 3D display 30 for the 3D display 30 to display the converted stereoscopic images.

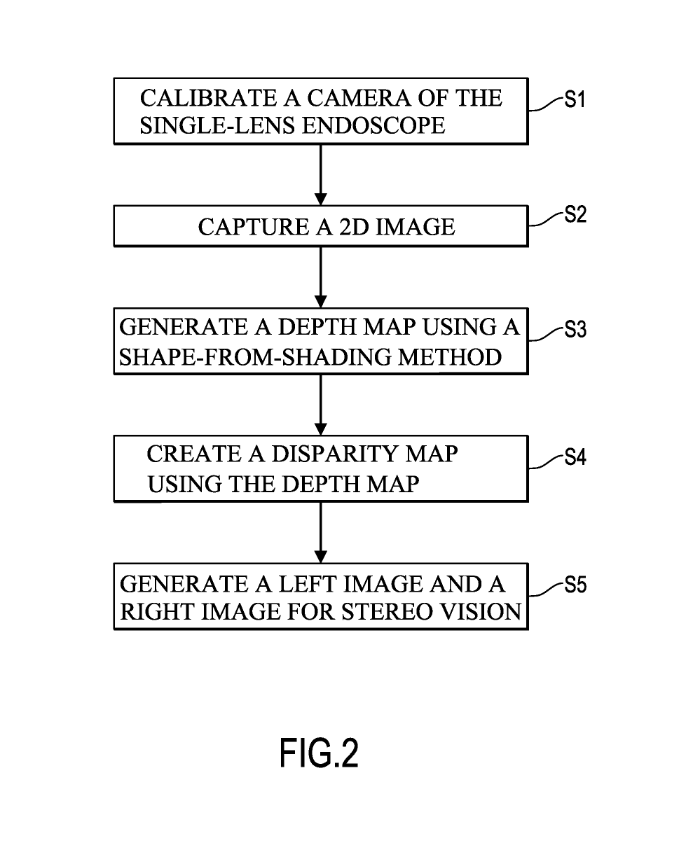

With reference to FIG. 2, a stereo display method for endoscope using shape-from-shading algorithm in accordance with the present invention is performed by the 2D-to-3D image conversion unit 12 to convert the 2D images from the monoscopic endoscope 20 into the stereoscopic images, and includes the following steps.

Step S1: Calibrate a camera of the monoscopic endoscope. With reference to “Image processing, analysis and machine vision, 2^{nd} edition, vol. 68, PWS, 1998, pp. 448-457”, a camera calibration method is used to calculate intrinsic parameters of the camera of the monoscopic endoscope. The camera calibration method estimates a camera posture by rotating and displacing a calibration template, and solves a nonlinear equation to obtain the intrinsic parameters and extrinsic parameters.

Step S2: Capture a 2D image. An image-capturing device is used to acquire a 2D image from the camera of the monoscopic endoscope. The image-capturing device may have a resolution being standard definition (SD) or high definition (HD). The camera of the monoscopic endoscope may have a 30 degree lens or a wide angle lens.

Step S3: Generate a depth map using shape-from-shading method. With reference to “Metric depth recovery from monocular images using shape-from-shading and specularities, Visentini-Scarzanella et al. 2012 IEEE Internal Conference on Image Processing”, a shape-from-shading method is employed to calculate lighting information and shading information of the 2D image generated from a light source, use an iterative approach to solve equations involving gradient variation of pixel information in the 2D image, and combine information associated with an illumination direction and a position of the light source to calculate a depth map of the pixels in the 2D image relative to the light source. An illumination position estimation of the light source disclosed in “Danail Stoyanov et al., 2009 IEEE/RSJ International Conference on Intelligent Robots and System (IROS), Illumination position estimation for 3D soft tissue reconstruction in robotic minimally invasive surgery” is provided to enhance accuracy in determining position of a light source. The pixel information of each pixel in the 2D image includes a pixel intensity value, the illumination direction and the natural logarithm of coordinates of the pixel. Fast sweeping methods disclosed in “Chiu-Yen Kao et al. SIAM J., Numerical Analysis 2005, Fast sweeping methods for static Hamilton-Jacobi equation” and parallel computation can be applied to speed up the iterative process.

The shape-from-shading method can be described by calculation of light distribution of a light source in the following.

Assume that a camera is located at C(α, β, γ), which can be pre-determined with the illumination position estimation. Given a set of coordinates of each pixel x=(x, y) in the 2D image, a surface normal n and a light vector I at a 3D point M corresponding to the pixel x can be represented as:

$$n = ( {u}_{x} , {u}_{y} - \frac{( x + α ) {u}_{x} + ( y + β ) {u}_{y} + u ( x )}{f + γ} )$$

$$I = ( x + α , y + β , f + γ )$$

where u(x) is the depth at point x and uₓ, u_{y} are the spatial derivatives. Hence, an image irradiance equation can be expressed as follows in terms of the proposed parametrisations of I and n without ignoring the distance attenuation term between the light source and surface reflection to solve a conventional Lambertian SFS (Shape-from-shading) model.

$$I ( x ) = ρ \frac{I \cdot n}{{γ}^{2}}$$

where ρ is a surface albedo.

After the substitution v=lnu is performed, a Hamiltonian, which is known as a spatial transformation between the position of the camera and the light source, can be obtained as follows:

$$H ( x , ∇ v ) = I ( x ) \frac{1}{ρ} \sqrt{( {v}_{x}^{2} + {v}_{y}^{2} + {𝒥 ( x , ∇ u )}^{2}} \cdot {Q ( x )}^{\frac{3}{2}}$$

$$where$$

$$\{ \begin{matrix}𝒥 ( x , ∇ u ) = \frac{{u}_{x} ( x + α ) + {u}_{y} ( y + β ) + 1}{f + γ} \\ Q ( x ) = {( x + α )}^{2} {( y + β )}^{2} + {( f + γ )}^{2}\end{matrix}$$

The depth map of the image caused by light distribution can thus be generated after iterations of calculation of the foregoing equations. As being almost the same, the light vector and the camera position vector can be simplified to be the same vector.

Step S4: Create a disparity map using the depth map. The depth map is composed of a gray-level image containing information relating to the distance of scene objects on the 2D image from a viewpoint. During the course of converting the depth map into a 3D stereo image pair, a disparity map is generated. Disparity values in the disparity map are inversely proportional to the corresponding pixel intensity values of the depth maps but are proportional to a focal length of a camera of the monoscopic endoscope and an interorbital width of a viewer.

Step S5: Generate a left image and a right image for stereo vision. The disparity map acquired during the course of converting the depth map into the 3D stereo image pair is used for generation of a left eye image and a right eye image. Each disparity value of the disparity map represents a distance between two corresponding points in the left eye image and the right eye image for generation of the left eye image and the right eye image associated with the 3D stereo image pair. The generated left eye image and right eye image can be further processed for various 3D display formats, such as side-by-side, interlaced and other 3D display formats, for corresponding 3D displays to display.

As can be seen from the foregoing description, the depth information can be calculated from the 2D image by using the shape-from-shading algorithm. After generation of the depth map, the 2D images can be combined with the depth maps to generate corresponding stereoscopic images without either replacing the conventional monoscopic endoscope with a dual-lens endoscope or altering the hardware structure of the conventional monoscopic endoscope. Accordingly, the issues arising from the conventional monoscopic endoscope providing no 3D stereo images and the costly dual-lens endoscope can be resolved.

Even though numerous characteristics and advantages of the present invention have been set forth in the foregoing description, together with details of the structure and function of the invention, the disclosure is illustrative only. Changes may be made in detail, especially in matters of shape, size, and arrangement of parts within the principles of the invention to the full extent indicated by the broad general meaning of the terms in which the appended claims are expressed.

What is claimed is:

1. A stereoscopic visualization system for endoscope using shape-from-shading algorithm, comprising:
a monoscopic endoscope capturing the two-dimensional (2D) images;
a three-dimensional (3D) display; and
an image conversion device connected between the monoscopic endoscope and the 3D display, and having:
an input port for endoscope connected to the monoscopic endoscope to receive the 2D image from the monoscopic endoscope;
a 2D-to-3D conversion unit applying shape from shading algorithm adapted to calculate a direction of a light source for the 2D image, and calculating a depth map based upon information of light distribution and shading of the 2D image, and applying depth image based rendering algorithm to convert the 2D image to a stereoscopic image with the information of light distribution and shading of the 2D image; and
an image output port connected with the 2D-to-3D image conversion unit and the 3D display to receive the stereo images and display the stereo image on the 3D display.

2. A stereo display method for endoscope using shape-from-shading algorithm, comprising steps of:
capturing a two-dimensional (2D) image, wherein an image-capturing unit is used to acquire a 2D image from a monoscopic endoscope with illumination from a light source;
calculating a light direction and a camera position for the 2D image;
generating a depth map of the 2D image using shape-from-shading method, wherein the shape-from-shading method combines the light direction and an iterative approach to solve equations involving a gradient variation of pixel intensity values in the 2D image; and
generating a stereoscopic image by combining the depth map and the 2D image.

3. The stereo display method as claimed in claim 2, wherein the shape-from-shading method is based on calculation of light distribution of a light source as follows:
assume that a camera is located at C(α, β, γ), which can be pre-determined with a illumination position estimation, a set of coordinates of each pixel x=(x, y) in the 2D image, a surface normal n and a light vector I at a 3D point corresponding to the pixel x of the 2D image are represented as:
$$n = ( {u}_{x} , {u}_{y} - \frac{( x + α ) {u}_{x} + ( y + β ) {u}_{y} + u ( x )}{f + γ} )$$
$$I = ( x + α , y + β , f + γ )$$
where u(x) is a depth at point x and uₓ, u_{y} are spatial derivatives;
an image irradiance equation is expressed as follows in terms of the light vector I and the surface normal n without ignoring distance attenuation between the light source and surface reflection to solve a Lambertian SFS (Shape-from-shading) model:
$$I ( x ) = ρ \frac{I \cdot n}{{γ}^{2}}$$
where ρ is a surface albedo;
after the substitution v=lnu is performed, a Hamiltonian, which is known as a spatial transformation between the position of the camera and the light source, is obtained as follows:
$$H ( x , ∇ v ) = I ( x ) \frac{1}{ρ} \sqrt{( {v}_{x}^{2} + {v}_{y}^{2} + {𝒥 ( x , ∇ u )}^{2}} \cdot {Q ( x )}^{\frac{3}{2}}$$
$$where$$
$$\{ \begin{matrix}𝒥 ( x , ∇ u ) = \frac{{u}_{x} ( x + α ) + {u}_{y} ( y + β ) + 1}{f + γ} \\ Q ( x ) = {( x + α )}^{2} {( y + β )}^{2} + {( f + γ )}^{2}\end{matrix}$$
the depth map of the 2D image caused by light distribution is generated after iterations of calculation of the foregoing equations, and the light vector and the camera position vector are simplified to be the same vector.

4. The stereo display method as claimed in claim 2, wherein the stereoscopic image is generated according to the depth image based rendering algorithm to provide different views of the 2D image with the 2D image and the depth map.
